(19) United States
(12) Patent Application Publication
(10) Pub. No.: US 2022/0218862 A1
(43) Pub. Date: Jul. 14, 2022

(54) ATOMIZATION DEVICE AND AROMA DIFFUSER

(71) Applicant: Puzhen Life Co., Ltd., Shatin (HK)

(72) Inventor: Andy LEE, Shatin (HK)

(73) Assignee: Puzhen Life Co., Ltd., Shatin (HK)

(21) Appl. No.: 17/189,705

(22) Filed: Mar. 2, 2021

(30) Foreign Application Priority Data
Jan. 14, 2021 (CN) 202120095054.6

(51) Int. Cl. A61L 9/14

(52) U.S. Cl. CPC A61L 9/14; A61L 2209/12; A61L 2209/11

(57) ABSTRACT

The present application provides an aroma diffuser, including: a body with an atomization port located on its upper surface; a touch screen, which is arranged on the upper surface of the body and configured to control an operation of the body. The touch screen has a ring shape and is arranged around the atomization port, the upper surface of the touch screen is a touch operation surface, and the cross-sectional shape of the touch operation surface is an arc shape concaved downwardly, a straight line shape inclined downwardly towards the atomization port or a curve shape inclined downwardly towards the atomization port.

### CROSS-REFERENCE TO THE RELATED APPLICATIONS

Pursuant to 35 U.S.C. §119 and the Paris Convention Treaty, this application claims the benefit of Chinese Utility Model Patent Application No. 202120095054.6 filed Jan. 14, 2021, the contents of which are incorporated herein by reference.

### TECHNICAL FIELD

The present application relates to the technical field of aromatherapy, and more particularly to an aroma diffuser.

### BACKGROUND

In the existing aroma diffuser, control buttons are arranged on a side of the main body, which is easy to be accidentally touched during a transportation process to trigger an operation of the body. In addition, the control buttons are arranged on the side of the body, which may not be easy to find the location of the control button due to the shelter of the body itself during use, and thus affect the convenience of operation.

### SUMMARY

An object of the present application is to overcome the above-mentioned shortcomings existed in the prior arts. In which an aroma diffuser is provided, aiming to solve the problem of inconvenient operation of the existing aroma diffuser.

In order to achieve the foregoing objective, embodiments of the present application provide the following technical solutions:

An aroma diffuser, including:

a body which has an atomization port disposed on an upper surface of the body;

a touch screen which is arranged on the upper surface of the body and configured to control an operation of the body, the touch screen is ring-shaped and arranged around the atomization port, an upper surface of the touch screen is configured as a touch operation surface, a cross-sectional shape of the touch operation surface is in an arc shape concaved downwardly, a straight line shape inclined downwardly towards the atomization port or a curve shape inclined downwardly towards the atomization port.

Optionally, the atomization port is located at a center position of the upper surface of the body, and the touch screen is a circular ring centered on the atomization port.

Optionally, the upper surface of the body has a first area and a second area, the touch screen is located in the first area, the atomization port is located in the second area, and the second area is recessed towards the atomization port.

Optionally, the touch operation surface has a plurality of touch keys for triggering the operation of the body, and a connection of each control button forms a circle around the center position of the upper surface of the body.

Optionally, the touch screen has a light transmissive structure at each touch key position.

Optionally, the touch screen is configured to enable all touch keys being displayed brightly when any touch key is touched by a human hand, and to turn off the bright display after a specific time.

Optionally, the touch operation surface has an atomization mode selection key and mode display signal lamps, the body has a plurality of atomization modes that can be changed upon triggering the atomization mode selection key, and each mode display signal lamp is configured to display a corresponding atomization mode information.

Optionally, the touch operation surface has an atomization time selection key and atomization time signal lamps, the body has a plurality of atomization operation time that can be changed upon triggering the atomization time selection key, each atomization time signal lamp is configured to display a corresponding atomization time information.

Optionally, the aroma diffuser further includes an illumination module, the illumination module includes a lamp body and an illumination key, the lamp body is arranged in the body, the illumination key is arranged in the touch screen and configured to control an operation of the lamp body.

Optionally, the aroma diffuser further includes a Bluetooth music module, the Bluetooth music module includes a Bluetooth receiver, a Bluetooth switch for turning on or off of the Bluetooth receiver, a music player and music keys for controlling an operation of the music player, the Bluetooth receiver and the music player are provided on the body, and the Bluetooth switch and the music keys are provided on the touch screen.

The aroma diffuser provided in this application adopts a touch screen to control the operation of the body, which can avoid the situation of misoperation caused by mechanical touch. The touch screen is arranged on the upper surface of the body which can facilitate operations. The touch operation surface is ring-shaped, and its cross-section is inclined or recessed towards the atomization port, which improves the aesthetics and reduces the risk of accidentally touching the control buttons during transportation or other situations.

### BRIEF DESCRIPTION OF DRAWINGS

In order to illustrate the technical solutions of embodiments of the present application more clearly, the drawings used in the description of the embodiments will be briefly introduced hereinbelow. Obviously, the drawings in the following description are merely some embodiments of the present application, and for persons skill in the art, other drawings can also be obtained on the basis of these drawings without any creative work.

FIG. 1 is a schematic diagram of a first structure of an aroma diffuser provided by an embodiment of the present application;

FIG. 2 is a partial cross-sectional view of the structure of FIG. 1;

FIG. 3 is a top view of the structure of FIG. 1;

FIG. 4 is a schematic diagram of a second structure of an aroma diffuser provided by an embodiment of the present application;

FIG. 5 is a partial cross-sectional view of the structure of FIG. 5;

FIG. 6 is a schematic diagram of a third structure of an aroma diffuser provided by an embodiment of the present application;

FIG. 7 is a partial cross-sectional view of the structure of FIG. 6.

Among them, reference signs in the figure are listed as follows:

10—body; 101—atomization port; 20—touch screen; 201—touch operation surface.

### DETAILED DESCRIPTION OF THE EMBODIMENTS

In order to illustrate technical problems to be solved, technical solutions and beneficial effects of the present application more comprehensible, the present application is further described in detail hereinbelow with reference to the drawings and embodiments. It should be understood that the specific embodiments described herein are merely used to explain the present application, and do not intend to limit the present application.

It should be noted that when an element is referred to as being “fixed to” or “disposed on” another element, it can be directly or indirectly on the other element. When an element is referred to as being “connected to” another element, it can be directly or indirectly connected to the other element.

It should be understood that the orientation or positional relationship indicated by the terms “upper”, “vertical”, “horizontal”, “inner”, “outer”, etc. are based on the orientation or positional relationship shown in the drawings, which are intended solely for the purpose of describing and simplifying this application, rather than indicating or implying a device or an element referred to must have a specific orientation, be constructed and operated in a specific orientation, and therefore cannot be understood as a limitation of the present application.

In addition, in the description of the present application, “a plurality of” means two or more than two, unless otherwise specifically defined.

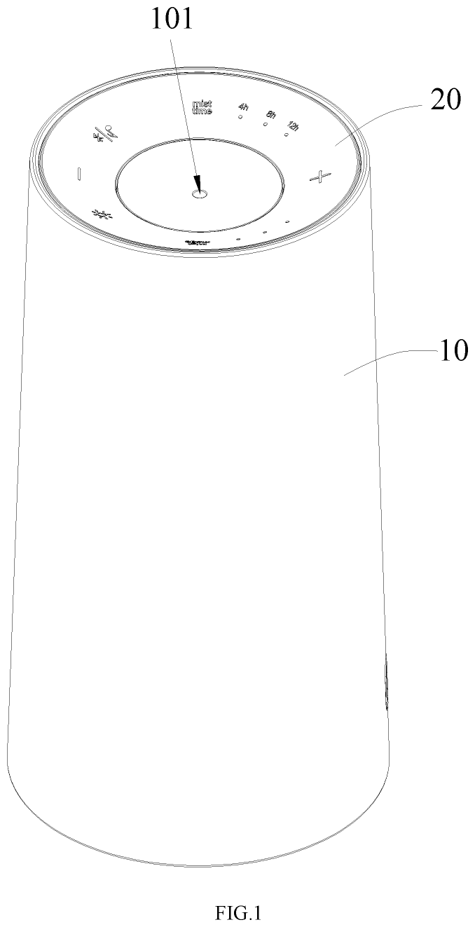

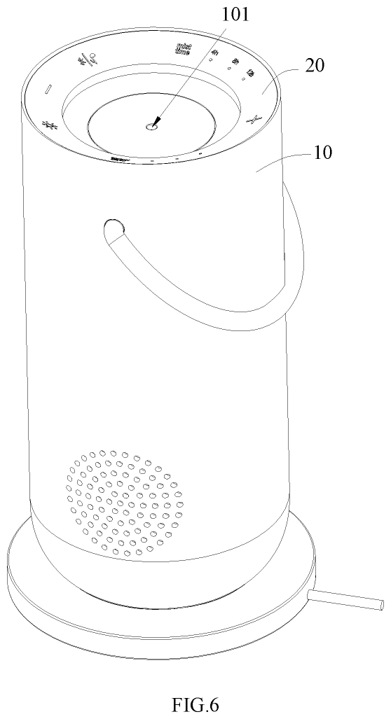

Please refer to FIG. 1 to FIG. 7 for an exemplary description of an aroma diffuser provided in the present application. The aroma diffuser includes a body 10 and a touch screen 20.

The body 10 has an atomization component inside, and the atomization component is configured to atomize essential oil and discharge it through an atomization port 101 located on an upper surface. It can be understood that if the essential oil is replaced with ordinary liquid water, the aroma diffuser in this embodiment becomes a humidifier, and the atomization component is configured to atomize the liquid water and discharge it through the atomizing port 101 located on the upper surface to achieve an effect of increasing the air humidity.

The touch screen 20 is provided on the upper surface of the body 10 and configured to control an operation of the body 10.

The touch screen 20 integrates control buttons for an operation of the atomization component, including atomization switch, atomization mode selection, atomization time selection, etc. Each control button may be a touch key, which can beautify the appearance and avoid misoperation caused by mechanical touch compared to mechanical buttons.

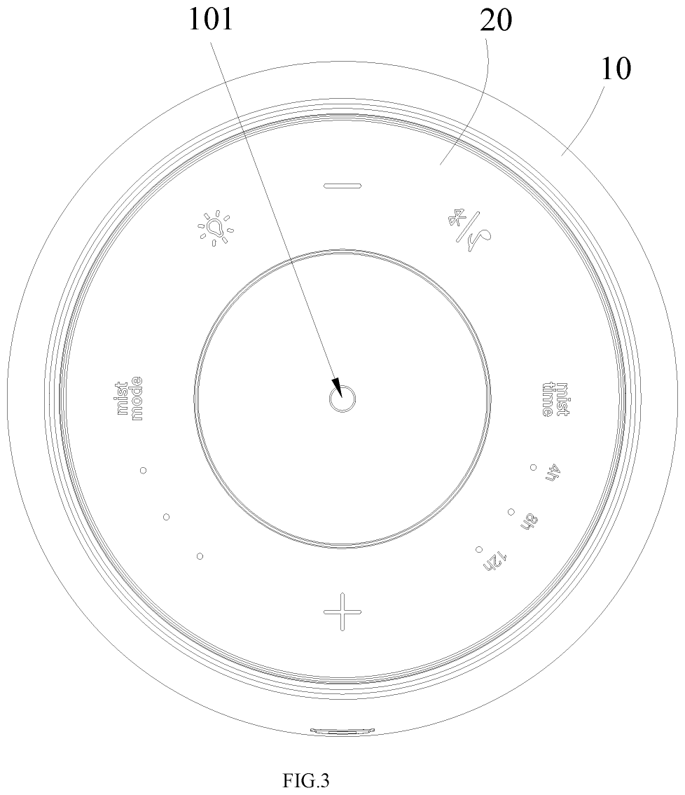

In the structure shown in FIG. 3, the touch keys include an atomization mode selection key (i.e., mist mode) and an atomization time selection key (i.e., mist time). A user may select an atomization mode by touching the atomization mode selection key with a finger to adjust an atomization speed. Three mode display signal lamps are provided next to the atomization mode selection key to visually display the atomization mode selected by the user. The user may select an atomization time by touching the atomization time selection key with a finger. In this embodiment, three atomization time options may be set in the aroma diffuser, i.e., 4 hours, 8 hours and 12 hours for atomization. Three atomization time signal lamps are provided next to the atomization time selection key and marked with text symbols to visually display the atomization time selected by the user.

The touch screen 20 has a ring shape and is arranged around the atomization port 101. The upper surface of the touch screen 20 is a touch operation surface 201. A cross-sectional shape of the touch operation surface 201 may be in an arc shape concaved downwardly, a straight line shape inclined downwardly towards the atomization port 101 or a curve shape inclined downwardly towards the atomization port 101.

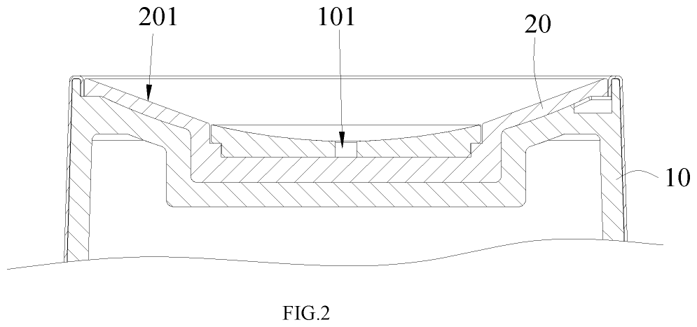

In the structure shown in FIG. 2, the cross-sectional shape of the touch operation surface 201 is a straight line inclined toward the atomization port 101. In this setting, the touch operation surface 201 is a conical surface of a circular cone with a wide top and a narrow bottom.

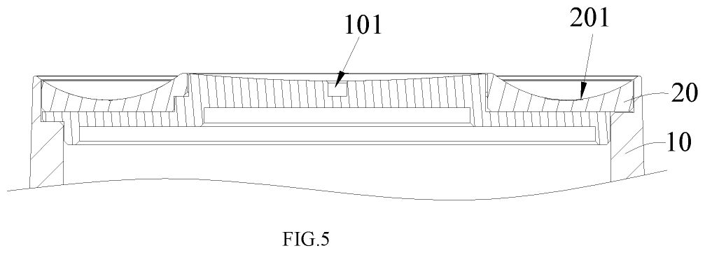

In the structure shown in FIG. 5, the cross-sectional shape of the touch operation surface 201 is in an arc shape concaved downwardly. Preferably, it is a circular arc. The touch keys are located at the lowest position of the circular arc.

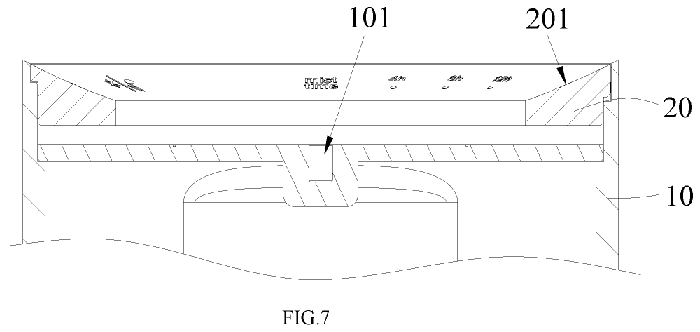

In the structure shown in FIG. 7, the cross-sectional shape of the touch operation surface 201 is in a curve shape inclined downwardly towards the atomization port 101.

The touch keys are distributed on the touch screen 20 and arranged along the circumference of the touch screen 20. In combination with use environment of the aroma diffuser, the touch keys are arranged on the upper surface of the body 10, which may not be easily blocked by the body 10 itself and are more intuitive to be seen, compared to be arranged on the side surface of the body 10, thus facilitating the convenience of operation.

The touch operation surface 201 is inclined toward the atomization port 101, or recessed downward, so that the touch keys are lower than the level of the upper surface of the aroma diffuser. In other words, the touch keys do not protrude from the upper surface of the aroma diffuser, thereby reducing a risk of accidentally touching the touch keys by human hands during transportation or other situations. On the other hand, the downwardly recessed or downwardly inclined arrangement conforms to the contour of a human finger, and improves an overall aesthetic effect without affecting the convenience of use.

In view of the above, the aroma diffuser provided by the present application adopts the touch screen 20 to operate and control the body 10, which can avoid the misoperation of mechanical touch. The touch screen 20 is arranged on the upper surface of the body 10 which facilitates operation. The touch operation surface 201 has a ring shaped, and its cross-section is inclined or recessed downwardly towards the atomization port 101, which improves the aesthetics and reduces the risk of accidentally touching the touch keys by human hands during transportation or other situations.

In another embodiment of the present application, the touch screen 20 has a light transmissive structure at the position of each touch key. The touch keys are provided with a luminous body, and the luminous body emits light to make each touch key brightly displayed when any touch key is triggered.

In this embodiment, the touch screen 20 is configured to enable all the touch keys being displayed brightly when any touch key is touched by a human hand and to turned off the bright display after a specific time. The touch screen 20 may not be easy to find the accurate position of each touch key in a dark environment such as night, thus the touch screen 20 is configured to make all touch keys brightly displayed when it is touched by a human hand for the first time, thereby facilitating the user to check the position of each touch key for subsequent control operations. It should be understood that the time for all the touch keys to light up can be 1 second, 2 seconds, 3 seconds, etc. After a preset time is reached, the bright display will be turned off automatically.

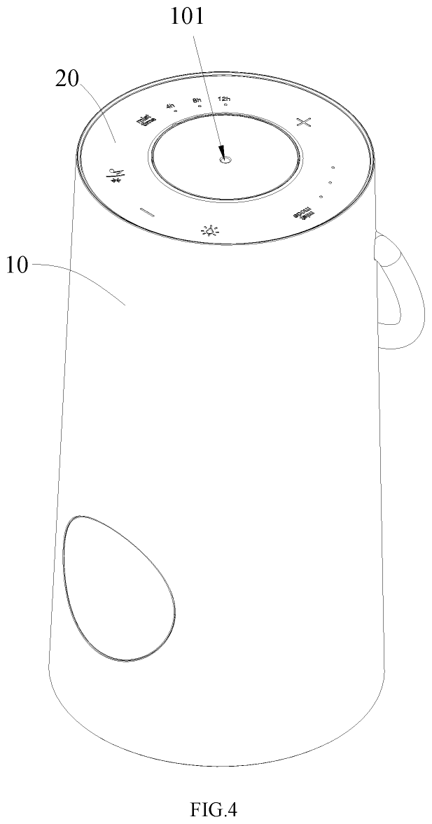

In another embodiment of the present application, please refer to FIG. 4, the aroma diffuser further includes an illumination module, the illumination module includes a lamp body and an illumination key, the lamp body is arranged in the body 10, and the illumination key is arranged on the touch screen 20 and used to control the operation of the lamp body. In this embodiment, the lamp body may be an atmosphere lamp, and an ON-FF or color change of the lamp body are controlled by touching the illumination key.

In another embodiment of the present application, please refer to FIG. 3. The aroma diffuser also includes a Bluetooth music module, which includes a Bluetooth receiver, a Bluetooth switch for turning on or off of the Bluetooth receiver, a music player and music keys for controlling the operation of the music player. The bluetooth receiver and the music player are arranged on the body 10, and the bluetooth switch and the music keys are arranged on the touch screen 20.

The music keys includes a music switch, a volume up key and a music down key. In the structure shown in FIG. 3, the music switch and the Bluetooth switch are integrated arranged. The volume up key and the music down key are separately provided on opposite sides of the touch operation surface 201.

In another embodiment of the present application, referring to FIGS. 1 and 3, the body 10 is in the shape of a truncated cone extending up and down, the atomization port 101 is located at a center position of the upper surface of the body 10, and the touch screen 20 may be a ring centered on the atomization port 101. In other embodiments, the body 10 may also has a columnar shape with a rectangular or other shape in cross section. Correspondingly, the touch screen 20 is a ring with rectangular or other shapes.

In another embodiment of the present application, referring to FIG. 3, the touch operation surface 201 has a plurality of touch keys for triggering the operation of the body 10, and the connection of each touch key forms a circle around the center position of the upper surface of the body 10.

The touch keys can be evenly distributed, or can be arranged according to function zones. Taking the direction shown in FIG. 3 as an example, the touch keys include the atomization mode selection key, the atomization time selection key, the music/Bluetooth switch key, the illumination key, the volume up key and the volume down key, the volume up key and the volume down key respectively located on the upper and lower sides of the atomization opening 101 which divides the touch operation surface 201 into two areas on the left and the right. The atomization mode selection key and the corresponding mode display signal lamps together serve as an atomization mode selection area, and are arranged on the left area of the touch operation surface 201 up and down with the illumination key. The atomization time selection key and the atomization time signal lamps together serve as an atomization time selection area, and are arranged on the right side of the touch operation surface 201 up and down with the music/Bluetooth switch key.

In another embodiment of the present application, referring to FIG. 2 or FIG. 5, the upper surface of the body 10 has a first area and a second area, the touch screen 20 is located in the first area, and the atomization port 101 is located in the second area. The second area is recessed towards the atomization port 101. It can be understood that the essential oil is discharged through the atomization port 101. During a discharging process of essential oil, the discharge of the essential oil lacks subsequent power, the essential oil tends to stick to the side of the atomization port 101 especially after the aroma diffuser is turned off. The second area is recessed towards the atomization port 101 to form a guide surface for guiding the essential oil to flow toward the atomization port 101, so that the essential oil stuck in the atomization port 101 is returned to the body 10.

In the structure shown in FIGS. 1 and 2, a level of the second area is located below the touch operation surface 201, and the upper surface of the first area and the touch operation surface 201 transition smoothly into a bowl shape.

In the structures shown in FIGS. 4 and 5, the second area protrudes from the first area so that the atomization port 101 and the touch key are substantially at a same horizontal position.

The foregoing description are merely preferred embodiments of the present application, and are not intended to limit the present application. Any modifications, equivalent substitutions and improvements made within the spirit and principles of this disclosure should be understood as being included within the protection scope of the present application.

What is claimed is:

1. An aroma diffuser, comprising:
a body having an atomization port disposed on an upper surface of the body;
a touch screen arranged on the upper surface of the body and configured to control an operation of the body, wherein the touch screen is ring-shaped and arranged around the atomization port, an upper surface of the touch screen is configured as a touch operation surface, and a cross-sectional shape of the touch operation surface is in an arc shape concaved downwardly, a straight line shape inclined downwardly towards the atomization port or a curve shape inclined downwardly towards the atomization port.

2. The aroma diffuser according to claim 1, wherein the atomization port is located at a center position of the upper surface of the body, and the touch screen is a circular ring centered on the atomization port.

3. The aroma diffuser according to claim 1, wherein the upper surface of the body is provided with a first area and a second area, the touch screen is located in the first area, and the atomization port is located in the second area, the second area is recessed towards the atomization port.

4. The aroma diffuser according to claim 1, wherein the touch operation surface is provided with a plurality of touch keys configured for triggering the operation of the body, and a connection of each touch key forms a circle around the center position of the upper surface of the body.

5. The aroma diffuser according to claim 4, wherein the touch screen has a light transmissive structure at each touch key position.

6. The aroma diffuser according to claim 5, wherein the touch screen is configured to enable all touch keys being displayed brightly when any touch key is touched by a human hand and to turn off the bright display after a specific time.

7. The aroma diffuser according to claim 1, wherein the touch operation surface is provided with an atomization mode selection key and mode display signal lamps, wherein the body has a plurality of atomization modes that can be changed upon triggering the atomization mode selection key, and each mode display signal lamp is configured to display a corresponding atomization mode information.

8. The aroma diffuser according to claim 7, wherein the touch operation surface is provided with an atomization time selection key and atomization time signal lamps, and the body has a plurality of atomization operation time that can be changed upon triggering the atomization time selection key, and each atomization time signal lamp is configured to display a corresponding atomization time information.

9. The aroma diffuser according to claim 8, wherein the aroma diffuser further comprises an illumination module, the illumination module includes a lamp body and an illumination key, the lamp body is arranged in the body, and the illumination key is arranged in the touch screen and configured to control an operation of the lamp body.

10. The aroma diffuser according to claim 8, wherein the aroma diffuser further comprises a Bluetooth music module, the Bluetooth music module comprises a Bluetooth receiver, a Bluetooth switch for turning on or off of the Bluetooth receiver, a music player and music keys for controlling an operation of the music player, the Bluetooth receiver and the music player are provided on the body, and the Bluetooth switch and the music keys are provided on the touch screen.
